(19) United States
(12) Patent Application Publication
(10) Pub. No.: US 2010/0150835 A1
(43) Pub. Date: Jun. 17, 2010

(54) Synthesis of [18F] Fluoromethyl Benzene Using Benzyl Pentafluorobenzenesulfonate

(76) Inventors: Bengt Langstrom, Uppsala (SE); Farhad Karimi, Mansfield, MA (US); Elisabeth Blom, Uppsala (SE)

Correspondence Address: GE HEALTHCARE, INC., IP DEPARTMENT 101 CARNEGIE CENTER, PRINCETON, NJ, 08540-6231, US

(21) Appl. No.: 12/526,515

(22) PCT Filed: Feb. 26, 2008
(86) PCT No.: PCT/US08/55003

§ 371 Date: Mar. 1, 2010

Related U.S. Application Data
(60) Provisional application No. 60/891,827, filed on Feb. 27, 2007.

(51) Int. Cl. A61K 51/00; C12N 15/63

(52) U.S. Cl. 424/1.73; 435/320.1

(57) ABSTRACT

The present invention discloses the reactivity of ponytail (“PT”) sulfonates as leaving groups in nucleophilic fluorination reactions. The results showed that using a pentafluorobenzenesulfonate precursor is a suitable leaving group for n. c. a. nucleophilic ¹⁸F-fluorination in synthesis of [¹⁸F]fluoromethyl benzene, wherein this is a suitable leaving group for 18F-labeling with moderate reactivity. The PT-precursor seems to be quite stable. In an attempt to purify the crude 18F-labeled product using fluorous solid phase extraction (F-SPE), the radio labeled impurities decreased significantly. This provides an opportunity for utilizing PT methodology in both simple and fast purification methods.

### FIELD OF THE INVENTION

The present invention allows for the investigation for the use of ponytail (“PT”)-sulfonates as leaving groups in direct ¹⁸F-fluorination reactions followed by F-SPE purification using [¹⁸F] fluoromethyl benzene as a model compound. The present invention further relates to a radiopharmaceutical composition of [¹⁸F] fluoromethyl benzene as well as a method of generating an image together with one or more pharmaceutically acceptable adjuvants, excipients or diluents. The present invention also relates to the use of [¹⁸F] fluoromethyl benzene for the manufacture of a radiopharmaceutical for use in a method of in vivo imaging. The present invention further relates to a method of monitoring the effect of treatment of a human or animal body with a drug to detect a wide variety of diseases where said method comprising administering to said body a compound such as [¹⁸F] fluoromethyl benzene.

### BACKGROUND OF THE INVENTION

Positron emission tomography (“PET”) is a non-invasive imaging technique which allows in vivo measurements and quantification of biological and biochemical process at the molecular level, and thus it is considered as a Molecular Imaging technique. Czermin J and Phelps M. Annu Rev Med 2002; 53: 89-112. PET is not only a valuable diagnostic tool in oncology, cardiology and neurology but is also becoming a valuable tool in nuclear medicine for drug development. Id. There are a number of positron emitting radionuclides of interest, such as ¹⁵O, ¹³N, ¹¹C, ¹⁸F, ⁷⁶Br, ¹²⁴I and metals like ⁶⁸Ga, ⁶⁹Cu and ⁶⁴Cu. They all have properties of interest for various applications, especially ¹¹C, ¹⁸F and the other halogens are of interest because of their properties in a synthetic labeling perspective. Additionally, ¹⁸F is of interest due to its physical properties. There are also a number of drugs containing one or more fluorine atoms. In some studies within drug development the need of specific radioactivity is less, for example in straightforward distribution studies, so in these cases F-exchange could be used as the labeling method.

In general, fluorine is a small atom with a very high electronegativity. Id. Covalently bound fluorine is larger than a hydrogen atom but occupying a smaller van der Waal's volume than a methyl, amino or hydroxyl group. Id. Fluorine substituent effects on pharmacokinetics and pharmacodynamics are very obvious. Eckelman W C. Nucl Med Bio 2002; 29: 777-782. Therefore, the replacement of a hydrogen atom or a hydroxy group by a fluorine atom is a strategy frequently applied in both PET tracer and drug developments. Id. The replacement of a hydrogen atom by a fluorine atom can alter the pKa, the dipole moments, lipophilicity, hydrogen bonding, the chemical reactivity, the oxidative stability, the chemical reactivity of neighboring groups or metabolic processes. Smart B. E. J Fluorine Chemistry 2001; 109: 3-11. The replacement of a hydroxyl group is based on the hypothesis that fluorine is a hydrogen acceptor like the oxygen of a hydroxyl group. Czermin J and Phelps M. Annu Rev Med 2002; 53: 89-112.

As regards of its use for PET, fluorine-18 has excellent nuclear properties such as low positron energy that results in low radiation dose, short maximum range in tissue and convenient half-life (t_{1/2}=109.7 min) considering distribution to other hospitals and performing longer acquisition protocols.

Furthermore, the application of radiolabelled bioactive peptides for diagnostic imaging is gaining importance in nuclear medicine. Biologically active molecules, which selectively interact with specific cell types, are useful for the delivery of radioactivity to target tissues. For example, radiolabelled peptides have significant potential for the delivery of radionuclides to tumours, infarcts, and infected tissues for diagnostic imaging and radiotherapy. ¹⁸F is the positron-emitting nuclide of choice for many receptor-imaging studies. Therefore, ¹⁸F-labelled bioactive peptides have great clinical potential because of their utility in PET to quantitatively detect and characterise a wide variety of diseases.

Radiolabeling of compounds with [¹⁸F]-fluoride can be achieved either by indirect displacement using fluoroalkylation agents or direct displacement of a leaving group. Using fluoroalkylation agents or direct displacement is not always convenient for all pharmaceutical substrates due to the formation of by-products, low yield, and the difficulties in purification processes.

Therefore, the aim of this invention is to develop fluorous chemistry also known as ponytail chemistry, (”PT″) in a no carrier added (“n.c.a.”) nucleophilic ¹⁸F-fluorination. Using PT chemistry offers simplifications of the overall process going from [¹⁸F]-fluoride in target water to pure radiopharmaceutical since the compounds containing the ponytail can easily be removed by SPE-purification where the SPE-matrix contains a ponytail matrix and would then be applied as an alternative to solid phase or surface based chemistry. The ponytail matrix disclosed herein is defined as any fluorous compound that is removed and purified from a reaction with a PT-precursor.

Discussion or citation of a reference herein shall not be construed as an admission that such reference is prior art to the present invention.

### SUMMARY OF THE INVENTION

Perfluoroalkyl sulfonates are not suitable leaving groups for n. c. a. nucleophilic ¹⁸F-fluorination for synthesis of [¹⁸F]fluoromethyl benzene. However, using the corresponding pentafluorobenzenesulfonate precursor has shown promising results and thus is a suitable leaving group for ¹⁸F-labeling with moderated reactivity. The ponytail (“PT”) PT-precursor seems to be quite stable for at least 4-6 months. In an attempt to purify the crude ¹⁸F-labeled product using fluoride-solid phase extraction (“F-SPE”), the radio labeled impurities decreased significantly by about 70%.

The present invention investigates the use of PT-sulfonates as leaving groups in direct ¹⁸F-fluorination reactions followed by F-SPE purification to form simple fluorous model compounds such as [¹⁸F]fluoromethyl benzene.

One embodiment of the present invention encompasses a method for radiofluorination comprising a reaction of the following compounds:

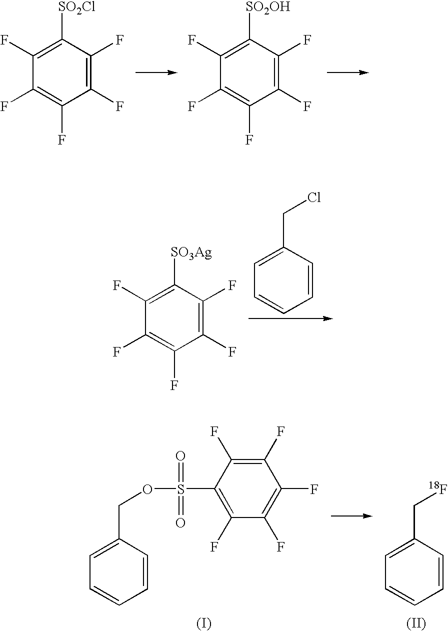

wherein (II) is purified using SPE, solid phase extraction.

### DETAILED DESCRIPTION OF THE INVENTION

Fluorous compounds contain a perfluoroalkyl group and virtually any molecule can have a fluorous analog. The perfluoroalkyl chain remains chemically inert during the reaction, while imparting unique properties to the reagents and sorbents during separation. These properties are due to a highly selective affinity (fluorous affinity interaction) between the reagent fluorous groups and the sorbent fluorous groups.

During separation, the chromatographic properties of the perfluoroalkyl group dominate the molecule's other functional groups. This critical property makes the organic domains of the fluorous molecules become chromatographically irrelevant to the fluorous sorbent. Hence the immense benefit of fluorous technology: diverse chemical structures containing the same fluorous group can be purified by simply using a single chromatographic method.

Fluorous Solid Phase Extraction (“F-SPE”) quickly separates fluorous compounds from non-fluorous compounds in three easy steps. First, the reaction mixture is loaded onto a chromatograph column. Second, the non-fluorous compounds are eluted with a fluorophobic solvent in one fraction. Third, the fluorous compounds are eluted with a fluorophilic solvent.

Furthermore, fluorous substrates are used to deliver a product that contains a fluorous tag. SPE can then be used to recover the individual, highly pure fluorous product from non-fluorous reagents. In the reverse approach, fluorous reagents can be used such that the byproducts are fluorous while the desired product is non-fluorous. Simple separation by F-SPE yields a high purity product.

The aim of the present invention is to develop fluorous chemistry, also known as ponytail (“PT”) chemistry, via n. c. a. nucleophilic ¹⁸F-fluorination. Using PT chemistry offers potential simplifications of the overall process going from [¹⁸F]-fluoride in target water to pure radio-pharmaceutical since the compounds containing the ponytail easily can be removed and the product purified using solid phase extraction where the SPE contains a ponytail matrix.

There are various advantages of using a solid phase extraction approach over conventional liquid synthesis approaches in labeling reactions.

One advantage in using a solid phase approach over conventional liquid synthesis in labeling reactions is the simplified kit-concept of using the solid phase approach i.e. direct ¹⁸F fluorination reactions. Another advantage is the easy cleanup in between consecutive reaction steps using the solid phase approach. Yet one other advantage of using the solid phase approach is the improved purification the solid phase approach delivers in labeling reactions in comparison. Still a further advantage of the present invention presents that the solid phase approach has a much easier automated process in comparison to the conventional liquid synthesis. Another advantage of the present invention's use of a solid phase approach depicts an improved yield of product through a time optimized process that is in comparison to other conventional synthesis.

One embodiment of the present invention depicts a method for radiofluorination comprising a reaction of a compound of formula (I) with a compound of formula (II) or benzyl bromide or benzyl iodide or any other halogen thereof where:

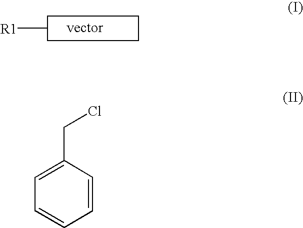

to give a compound of formula (III):

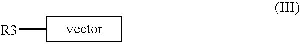

where
R1 is SO₂Cl, SO₂Br, or SO₂I attached to said vector and then SO₂Cl, SO₂Br, or SO₂I attached to said vector are treated with water to form SO₂OH attached to said vector and next SO₂OH attached to said vector are treated with silver carbonate to form SO₃Ag attached to said vector
R3 is

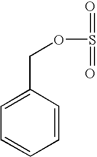

to give formula (IV):

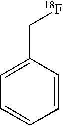

wherein formula (IV) is purified with SPE and contains a ponytail matrix.

A further embodiment of the present invention shows a method according to the above scheme wherein the vector comprises:

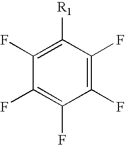

and where R1 can be attached to any of the carbons on the benzene ring or any of the attached fluorine atoms can be attached at any place along the benzene ring.

Another embodiment of the present invention encompasses a method for radiofluorination comprising a reaction of the following compounds:

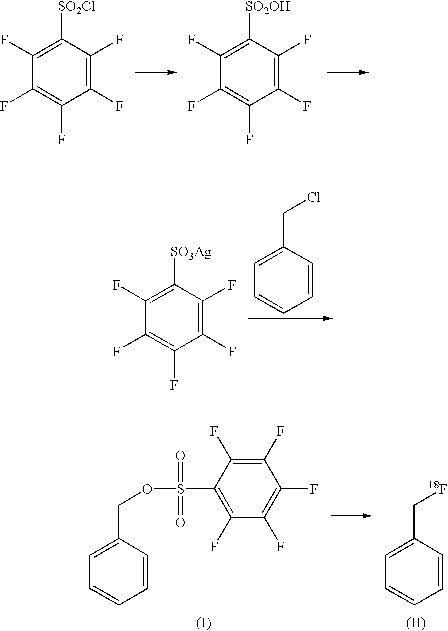

wherein (II) is purified using SPE, solid phase extraction and contains a ponytail matrix.

A vector used herein is a fragment of a compound or moiety having affinity for a receptor molecule. An example of such a vector used herein comprises a pentafluorobenzene structure.

A further embodiment of the present invention depicts the SPE contains a ponytail matrix. The present invention shows that the SPE occurs at least twice as fast as conventional liquid synthesis processes. As mentioned earlier, the ponytail matrix disclosed herein is defined as any fluorous compound that is removed and purified from a reaction with a PT-precursor.

Still another embodiment of the present invention shows a radiopharmaceutical composition comprising an effective amount of a compound of formula (IV); together with one or more pharmaceutically acceptable adjuvants, excipients or diluents.

Another further embodiment of the present invention depicts a method of generating an image of a human or animal body comprising administering a compound of formula (IV) to said body and generating an image of at least a part of said body to which said compound is distributed using positron emission tomography (“PET”). PET is a type of nuclear medicine imaging. Nuclear medicine imaging procedures are noninvasive and usually painless medical tests that help physicians diagnose medical conditions. These imaging scans use radioactive materials such as [¹⁸F] fluoromethyl benzene.

A further embodiment of the present invention depicts the use of a compound of formulas (IV) for the manufacture of a radiopharmaceutical for use in a method of in vivo imaging.

Yet another embodiment of the present invention shows a method of monitoring the effect of treatment of a human or animal body with a drug to combat a condition associated with cancer, preferably angiogenesis, said method comprising administering to said body a compound of formulas (X and (Y) and detecting the uptake of said conjugate by cell receptors said administration and detection optionally but preferably being effected before, during and after treatment with said drug.

### Examples

The invention is further described in the following examples, which is in no way intended to limit the scope of the invention.

The invention is illustrated by way of examples in which the following abbreviations are used:
hr(s): hour(s)
min(s): minute(s)
Bn: benzyl group
Ph: phenyl
Me: methyl
RT: room temperature
SPE: solid phase extraction
Benzyl chloride:
Pentafluorobenzenesulfonate:
CH2Cl_{2:} methyl chloride:
KHPO₄:
MeCN: methyl cyanide

### Precursor Synthesis

Proof of concept in this study was obtained using compound (A), Scheme 1. 2,3,4,5,6-pentafluoro-benzenesulfonyl chloride was treated with water followed by silver carbonate. The resulted silver salt was reacted with benzyl chloride as shown in Scheme 1.

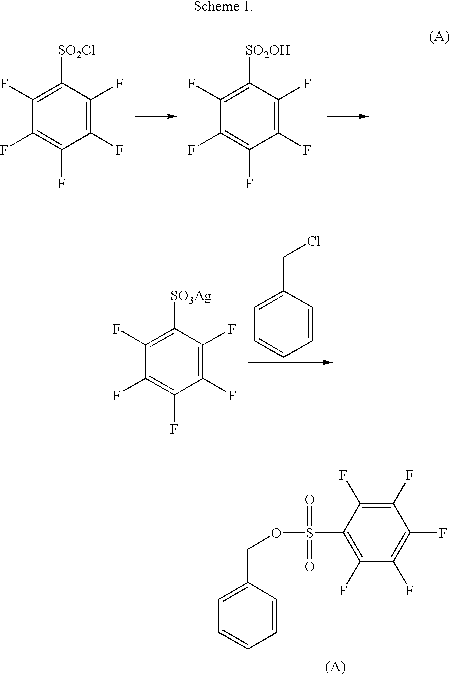

Method for Preparing the Precursor benzyl pentafluorobenzenesulfonate (A)

2,3,4,5,6-pentafluoro-benzenesulfonyl chloride (3.030 grams, 11.37 millimoles) was added to 8 milliLiters H₂O. The reaction mixture was heated at 100° Centigrade for 22 hours and thereafter concentrated under reduced pressure. The resulting 2,3,4,5,6-pentafluoro-benzenesulfonic acid was redissolved in 10 mL H₂O and silver carbonate (3.125 grams, 11.33 millimoles) was added. After stirring the reaction mixture for 25 hours at room temperature in darkness excess silver carbonate was filtered off and the filtrate was concentrated under reduced pressure. The resulting silver salt was dissolved in 9 milliLiters dry acetonitrile and benzyl chloride (1.301 grams, 10.28 millimole) was added. Thereafter the mixture was stirred at 85° Centigrade in darkness for 17 hours and concentrated under reduced pressure. The residue was purified by column chromatography (100% CH2Cl₂) yielding I as yellow crystals (0.350 grams, 10%).

### Radiochemistry

The materials setforth below were used to obtain radio-labeled compounds with [¹⁸F] fluoride.

1 Water (95%) enriched in ¹⁸O;

2 QMA Accell Plus quaternary methylammonium anion-exchange resin;

3 Kryptofix 2.2.2;

4 Anhydrous potassium carbonate;

5 Anhydrous acetonitrile;

6 The corresponding precursor such as compound (I);

7 Millipore Millex GV sterilizing filter;

8 Glass reaction vessels: ReactiVials (5 ml) from Altech;

9 Analytical column: Discovery ODS.5 ␣m 250 mm×4.6 mm; and

10 F-SPE, FluoroFlash®, (Si(CH₂)₂C₈F₁₇)).

### Analytical HPLC Methods Used:

Linear gradient elution of 40% KHPO₄ (25 mM) and 60% MeCN/H₂O (50:7) to 10% KHPO₄ (25 mM) and 90% MeCN/H₂O (50:7) for 5 minutes with a flow rate 1.5 milliliter/minute.

Sample Preparation:

An analytical sample was prepared from reaction mixture in 70% Ethyl Alcohol (EtOH).

### ¹⁸F Production

[¹⁸F] Fluoride was produced at Uppsala Imanet by an ¹⁸O(p, n) ¹⁸F nuclear reaction through proton irradiation of enriched (95%) ¹⁸O water using Scanditronix MC-17 cyclotron.

Method for Preparing ¹⁸F-Labeling Benzene (B) Using Precursor Benzyl pentafluorobenzenesulfonate (A)

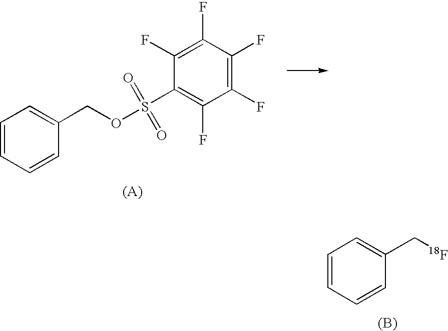

A solution of benzyl pentafluorobenzenesulfonate (5.0 milligrams) in 0.2 milliliter of acetonitrile was added to a dry residue containing the complex [Kryptofix/Kryptofix 2.2.2]^{+ 18}F in 0.2 millilitter of acetonitrile. The reaction was performed in a closed vessel at 150° C. for 15 minutes.

The results using precursor A, containing pentafluorobenzenesulfonate, showed that this is one suitable leaving group for n. c. a. nucleophilic ¹⁸F-fluorination. The possibilities for fluorous SPE purification methods was illustrated using FluoroFlash® which in using this example gave a substantial purification of the labeled product.

Furthermore, the solid phase extraction is applicable in essentially all areas from traditional synthesis through parallel synthesis, and is especially useful for parallel synthesis of intermediates.

The PT-precursor seems to be stable for at least 4-6 months. New PT-precursors should be synthesized for exploring the scope and limitation of this methodology. This example is a proof of concept for the idea of using suitable perfluoro-substituted leaving groups combined with fast Fluorous SPE purification approaches.

### F-SPE Conditions:

2 g SPE-column (FluoroFlash®, (Si(CH₂)₂C₈F₁₇)).
1) The cartridge was washed with 1 ml DMF, all DMF pushed out.
2) Preconditioning with 2 ml 80:10 MeOH:H₂O, all MeOH:H₂O pushed out.
3) Reaction mixture loaded. All solvent pushed out.
4) Fluorophobic elution: 2 ml 80:10 MeOH:H₂O, all MeOH:H₂O pushed out.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for radiofluorination comprising a reaction of a compound of formula (I) with a compound of formula (II) or benzyl bromide or benzyl iodide: 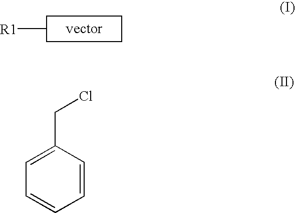
to give a compound of formula (III): 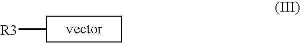
where
R1 is SO₂Cl, SO₂Br, or SO₂I attached to said vector and then SO₂Cl, SO₂Br, or SO₂I attached to said vector are treated with water to form SO₂OH attached to said vector and next SO₂OH attached to said vector are treated with silver carbonate to form SO₃Ag attached to said vector
R3 is 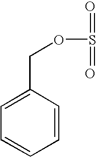
to give formula (IV): 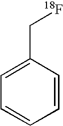
wherein formula (IV) is purified with SPE and contains a ponytail matrix.

2. A method according to claim 1 wherein the vector comprises: 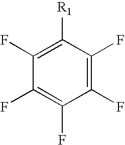
where R1 can be attached to any of the carbons on the benzene ring.

3. A radiopharmaceutical composition comprising an effective amount of a compound of formula (IV) according to claim 1; together with one or more pharmaceutically acceptable adjuvants, excipients or diluents.

4. A method of generating an image of a human or animal body comprising administering a compound of formula (IV) according to claim 1 to said body and generating an image of at least a part of said body to which said compound is distributed using PET.

5. Use of a compound of formula (IV) according to claim 1 for the manufacture of a radiopharmaceutical for use in a method of in vivo imaging.

6. A method of monitoring the effect of treatment of a human or animal body with a drug to combat a condition associated with tumours, infarcts, and infected tissues said method comprising administering to said body a compound of formula (IV) according to claim 1 and detecting the uptake of said compound by cell receptors said administration and detection optionally but preferably being effected before, during and after treatment with said drug.
